(19) United States
(12) Patent Application Publication
(10) Pub. No.: US 2026/0134981 A1
(43) Pub. Date: May 14, 2026

(54) HUMAN-IN-THE-LOOP CLINICAL OVERRIDE AND ACCOUNTABILITY GOVERNANCE SYSTEM FOR AI-ASSISTED MEDICAL DECISION SUPPORT

(71) Applicant: George William Bickerstaff, III, Greenwich, CT (US)

(72) Inventor: George William Bickerstaff, III, Greenwich, CT (US)

(21) Appl. No.: 19/445,559

(22) Filed: Jan. 11, 2026

(51) Int. Cl. G16H 40/20; H04L 9/32

(52) U.S. Cl. CPC G16H 40/20; H04L 9/32; H04L 2209/88

(57) ABSTRACT

A human-in-the-loop clinical override and accountability governance system enforces explicit clinician evaluation of AI-assisted clinical outputs prior to workflow execution. The system generates cryptographically protected accountability records binding clinician judgment to AI-assisted outputs, improving governance, safety, and trust without autonomously diagnosing conditions or prescribing treatment actions.

### TECHNICAL FIELD

The present invention relates to computer-implemented governance and accountability systems for artificial intelligence-assisted clinical decision support and, more particularly, to execution-time human-in-the-loop override, responsibility attribution, and cryptographically verifiable accountability control applied to AI-assisted clinical workflows without autonomously diagnosing medical conditions or prescribing treatment actions.

### BACKGROUND

Artificial intelligence systems are increasingly integrated into clinical environments to assist healthcare professionals by generating alerts, prioritizations, recommendations, risk indicators, or workflow suggestions derived from patient data and operational context.

While such systems may enhance situational awareness and efficiency, they introduce systemic risk when AI outputs are accepted, modified, or rejected without explicit documentation of human judgment, authority, and responsibility at the point of clinical decision-making.

Existing clinical information systems frequently lack technical mechanisms that require, capture, and preserve explicit clinician override decisions, structured justification, identity attribution, and immutable accountability records in response to AI-assisted outputs.

This absence of deterministic accountability undermines patient safety, regulatory compliance, medico-legal review, institutional governance, and post-event analysis, particularly in high-risk or time-sensitive clinical environments.

Accordingly, there exists a need for a technical governance control system that enforces human-in-the-loop authority over AI-assisted clinical outputs, captures cryptographically verifiable accountability records at execution time, and preserves clinician judgment as the final decision authority without autonomously diagnosing conditions or directing patient treatment.

### SUMMARY

The invention provides a computer-implemented human-in-the-loop clinical override and accountability governance system that receives outputs generated by artificial intelligence clinical decision support systems and enforces explicit clinician evaluation prior to workflow execution.

The system requires a clinician to accept, override, defer, or annotate each AI-assisted output and generates a cryptographically protected accountability record that binds the clinician's action, identity, timing, contextual metadata, and workflow state into an immutable governance artifact.

The system enforces execution-time accountability by preventing workflow progression absent a recorded clinician action and preserves regulator-ready evidence demonstrating that human judgment was applied to AI-assisted outputs prior to clinical action.

The system operates strictly as a governance and accountability control layer and does not autonomously diagnose medical conditions, prescribe treatments, or replace clinician authority.

### DEFINITIONS (ALPHABETICAL ORDER)

Accountability Record: An immutable, cryptographically protected data structure capturing clinician actions, identity, timing, contextual metadata, and workflow state associated with an AI-assisted output.
AI Clinical Decision Support System: A computational system that generates clinical support outputs without autonomously diagnosing medical conditions or prescribing treatments.
Clinician Override Action: An explicit clinician-initiated action to accept, modify, defer, or reject an AI-assisted output.
Contextual Annotation: Clinician-provided explanatory information associated with an override or acceptance action.
Execution-Time Governance: Enforcement of human authorization requirements before an AI-assisted output may influence a clinical workflow.
Human-in-the-Loop: A system design requiring affirmative human judgment prior to workflow execution.
Override Interface: A user interface through which clinicians review and act upon AI-assisted outputs.
Responsibility Assignment: Cryptographic association of a clinician identity with an action taken on an AI-assisted output.
Structured Justification: Predefined data fields used to capture standardized override rationale.
Workflow Item: A system-managed representation of an AI-assisted output.
Workflow State: A defined execution status governing how a workflow item may proceed.

### BRIEF DESCRIPTION OF THE DRAWINGS

FIG. 1 illustrates a human-in-the-loop clinical override and accountability governance architecture.

FIG. 2 illustrates AI output presentation and clinician evaluation.

FIG. 3 illustrates override capture and responsibility assignment.

FIG. 4 illustrates accountability record generation and cryptographic integrity protection.

FIG. 5 illustrates audit, reporting, and compliance outputs.

### DETAILED DESCRIPTION OF THE DRAWINGS

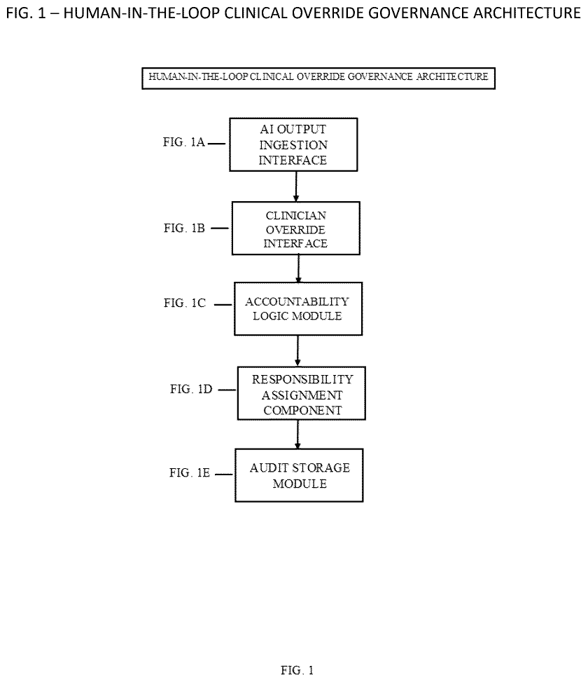

### FIG. 1—Human-in-the-Loop Clinical Override Governance Architecture

FIG. 1 illustrates an execution-time human-in-the-loop governance architecture for AI-assisted clinical decision support. The architecture separates AI output generation from clinical authority by inserting a mandatory clinician evaluation and accountability layer prior to workflow execution. Governance enforcement ensures that no AI-assisted output influences a clinical workflow without explicit human judgment and recorded responsibility.

### FIG. 1A—AI Output Ingestion Interface

FIG. 1A illustrates an interface configured to receive AI-assisted outputs from one or more clinical decision support systems. The interface normalizes outputs for consistent downstream handling without modifying underlying AI inference. Each output is encapsulated as a workflow item subject to governance controls.

### FIG. 1B—Clinician Override Interface

FIG. 1B illustrates a clinician-facing interface that presents AI-assisted outputs for review prior to execution. The interface requires an explicit clinician action before workflow progression is permitted. User interaction events are captured as part of the accountability process.

### FIG. 1C—Accountability Logic Module

FIG. 1C illustrates logic configured to capture clinician actions, identity attributes, timing, and contextual workflow data. The module coordinates enforcement of execution-time governance requirements. Captured data is forwarded for accountability record generation.

### FIG. 1D—Responsibility Assignment Component

FIG. 1D illustrates a component that cryptographically binds clinician identity to a specific action taken on an AI-assisted output. The binding establishes non-repudiable responsibility attribution. Once recorded, responsibility assignments cannot be altered.

### FIG. 1E—Audit Storage Module

FIG. 1E illustrates an immutable audit storage module that persists accountability records. The module enforces append-only storage semantics to prevent tampering. Stored records support downstream audit, compliance, and review processes.

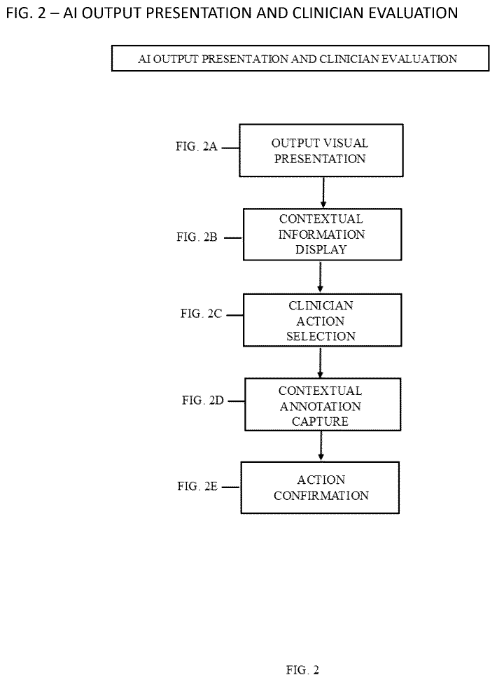

### FIG. 2—AI Output Presentation and Clinician Evaluation

FIG. 2 illustrates presentation of AI-assisted outputs for clinician evaluation prior to workflow execution. The evaluation process ensures that human judgment is applied before any clinical action is influenced. Workflow progression is suspended until evaluation is complete.

### FIG. 2A—Output Visual Presentation

FIG. 2A illustrates visual presentation of an AI-assisted output to a clinician. The presentation conveys relevant information without asserting clinical authority. Displayed content remains advisory in nature.

### FIG. 2B—Contextual Information Display

FIG. 2B illustrates presentation of contextual information associated with the AI-assisted output. Context may include patient state, timing, or system metadata. This information supports informed clinician judgment.

### FIG. 2C—Clinician Action Selection

FIG. 2C illustrates explicit clinician action controls allowing acceptance, override, or deferral of an AI-assisted output. Each action represents a discrete governance decision. Selection of an action initiates accountability capture.

FIG. 2D—Contextual Annotation Capture

FIG. 2D illustrates capture of structured and free-text clinician annotations. Annotations provide explanatory context for override or acceptance decisions. Captured annotations are incorporated into accountability records.

### FIG. 2E—Action Confirmation

FIG. 2E illustrates confirmation of the clinician's selected action. Confirmation finalizes the governance decision for the workflow item. Execution proceeds only after confirmation is recorded.

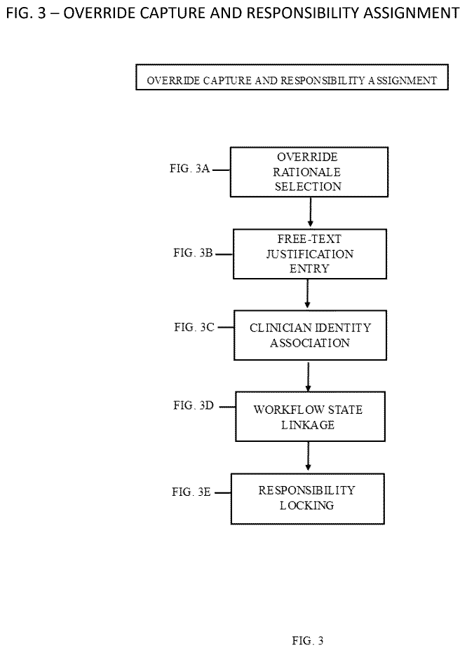

FIG. 3—Override Capture and Responsibility Assignment

FIG. 3 illustrates structured capture of clinician override decisions and responsibility attribution. The system enforces deterministic association between actions and actors. Responsibility is finalized prior to workflow execution.

### FIG. 3A—Override Rationale Selection

FIG. 3A illustrates selection of predefined rationale categories for override actions. Structured rationale enables consistent governance analysis. Selected categories are stored as part of accountability records.

### FIG. 3B—Free-Text Justification Entry

FIG. 3B illustrates entry of clinician-provided free-text justification. The entry captures situational reasoning not represented by predefined categories. Text entries are preserved immutably.

FIG. 3C—Clinician Identity Association

FIG. 3C illustrates cryptographic association of clinician identity with the override action. Identity verification ensures that responsibility attribution is accurate. The association is bound to the workflow item.

### FIG. 3D—Workflow State Linkage

FIG. 3D illustrates linkage of clinician actions to the current workflow state. The linkage ensures temporal and contextual accuracy of accountability records. State transitions are recorded deterministically.

### FIG. 3E—Responsibility Locking

FIG. 3E illustrates finalization and locking of responsibility assignments. Once locked, responsibility records cannot be modified or reassigned. This enforces non-repudiation.

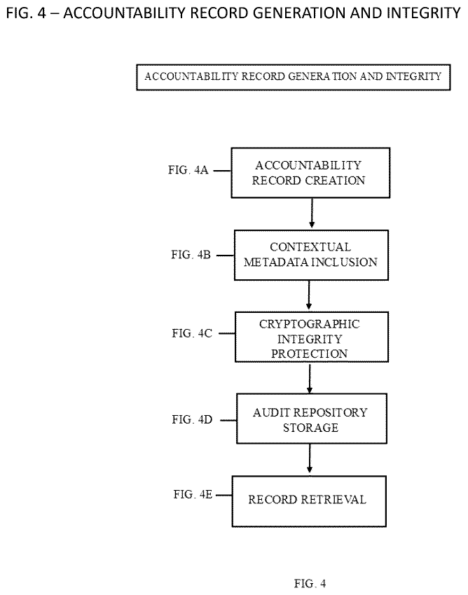

### FIG. 4—Accountability Record Generation and Integrity

FIG. 4 illustrates creation, protection, and storage of accountability records generated from clinician actions. Records are designed to be regulator-ready and tamper-resistant. Cryptographic integrity ensures long-term trustworthiness.

### FIG. 4A—Accountability Record Creation

FIG. 4A illustrates automatic generation of an accountability record upon clinician action confirmation. The record aggregates identity, action type, timing, and workflow context. Record creation occurs prior to workflow execution.

### FIG. 4B—Contextual Metadata Inclusion

FIG. 4B illustrates inclusion of system and workflow metadata within accountability records. Metadata supports audit reconstruction and compliance review. Inclusion is performed automatically.

### FIG. 4C—Cryptographic Integrity Protection

FIG. 4C illustrates application of cryptographic hashing and signing to accountability records. Integrity protection prevents unauthorized modification. Protected records support non-repudiation.

### FIG. 4D—Audit Repository Storage

FIG. 4D illustrates storage of protected accountability records in an immutable audit repository. The repository enforces append-only semantics. Records remain available for long-term review.

### FIG. 4E—Record Retrieval

FIG. 4E illustrates controlled retrieval of accountability records. Retrieval is subject to access controls and logging. Retrieved records preserve cryptographic integrity.

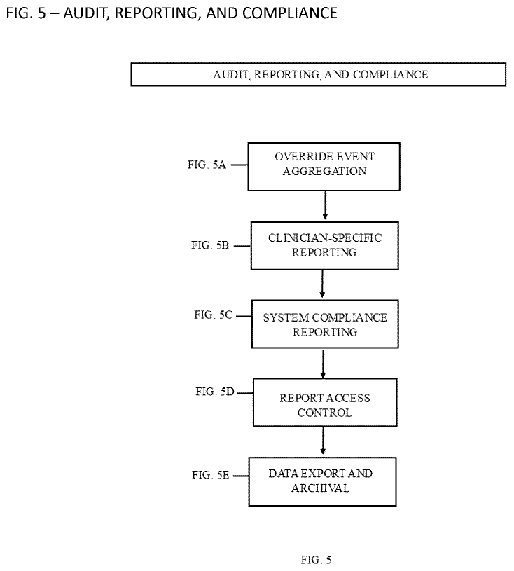

### FIG. 5—Audit, Reporting, and Compliance

FIG. 5 illustrates audit, reporting, and compliance outputs derived from accountability records. Aggregated views support institutional governance. Outputs do not alter clinical decision authority.

FIG. 5A—Override Event Aggregation

FIG. 5A illustrates aggregation of override and acceptance events across workflows. Aggregation supports trend analysis and governance review. Individual accountability remains preserved.

### FIG. 5B—Clinician-Specific Reporting

FIG. 5B illustrates generation of clinician-specific accountability reports. Reports summarize actions without altering responsibility attribution. Access is restricted to authorized roles.

### FIG. 5C—System Compliance Reporting

FIG. 5C illustrates institution-level compliance reporting derived from accountability data. Reports support regulatory and internal review. Reporting does not influence real-time workflows.

### FIG. 5D—Report Access Control

FIG. 5D illustrates enforcement of role-based access control for audit reports. Access events are logged for accountability. Unauthorized access is prevented.

### FIG. 5E—Data Export and Archival

FIG. 5E illustrates export and archival of accountability data. Archived records preserve integrity for long-term retention. Export supports external audit requirements.

### EXAMPLE

In one example, an AI system generates a clinical alert indicating potential neurological deterioration. A clinician reviews the alert through the override interface and elects to override the suggestion based on bedside assessment.

The system records the override action, clinician identity, timestamp, workflow state, and explanatory annotation and generates a cryptographically protected accountability record prior to workflow execution.

Subsequent audit reports summarize override frequency and rationale for governance review without autonomously diagnosing conditions or prescribing treatment actions.

1. A computer-implemented system comprising one or more processors and non-transitory memory storing instructions that cause the system to receive an AI-assisted clinical output, present the output for clinician evaluation, require an explicit clinician action prior to workflow execution, and generate an immutable accountability record binding clinician identity, action type, timing, and workflow state, wherein the system does not autonomously diagnose a medical condition or prescribe treatment.

2. A computer-implemented method comprising receiving an AI-assisted clinical output, presenting the output for clinician evaluation, receiving an explicit clinician action, cryptographically associating the action with clinician identity and workflow state, and storing an immutable accountability record prior to workflow execution.

3. A non-transitory computer-readable medium storing instructions that
cause one or more processors to perform the method of claim 2.

4. The system of claim 1, wherein accountability records include structured justification fields.

5. The system of claim 1, wherein contextual annotation is required for override actions.

6. The system of claim 1, wherein responsibility assignment is immutable.

7. The system of claim 1, wherein clinician identity is cryptographically verified.

8. The system of claim 1, wherein accountability records are protected against modification.

9. The method of claim 2, wherein override metrics are aggregated for governance reporting.

10. The system of claim 1, wherein audit reports are role-restricted.
